(19) United States
(12) Patent Application Publication
(10) Pub. No.: US 2021/0196170 A1
(43) Pub. Date: Jul. 1, 2021

(54) INFORMATION PROCESSING DEVICE AND NON-TRANSITORY COMPUTER READABLE MEDIUM

(71) Applicant: FUJI XEROX CO., LTD., Tokyo (JP)

(72) Inventors: Akira ICHIBOSHI, Kanagawa (JP); Kazunari KOMATSUZAKI, Kanagawa (JP); Ryota MIZUTANI, Kanagawa (JP); Shingo UCHIHASHI, Kanagawa (JP)

(73) Assignee: FUJI XEROX CO., LTD., Tokyo (JP)

(21) Appl. No.: 16/889,141

(22) Filed: Jun. 1, 2020

(30) Foreign Application Priority Data
Dec. 25, 2019 (JP) 2019-233897

(51) Int. Cl. A61B 5/16; G06N 20/00; A61B 5/11; A61B 5/024; A61B 5/00

(52) U.S. Cl. CPC A61B 5/165; G06N 20/00; A61B 5/1123; A61B 2562/0219; A61B 5/7264; A61B 5/4035; A61B 5/02416

(57) ABSTRACT

An information processing device is provided with a processor configured to output an evaluation value of stress accumulated in an observation period on a basis of information indicating a first stress feature accumulated up to before the observation period and a second stress feature received during the observation period.

### CROSS-REFERENCE TO RELATED APPLICATIONS

This application is based on and claims priority under 35 USC 119 from Japanese Patent Application No. 2019-233897 filed Dec. 25, 2019.

### BACKGROUND

### i) Technical Field

The present disclosure relates to an information processing device and a non-transitory computer readable medium.

### (ii) Related Art

A stress evaluation method that acquires biological data from a wearable sensor worn by a measurement subject and evaluates the stress felt by the measurement subject on the basis of the biological data has been proposed (for example, see

https://www.jstage.jst.go.jp/article/pjsai/JSAI2018/0/JSAI20 18_2F3OS4b05/_pdf).

The stress evaluation method described above (https://www.jstage.jst.go.jp/article/pjsai/JSAI2018/0/JSAI2 018_2F3OS4b05/_pdf) carries out a stress-related survey once a month on measurement subjects, collects acceleration (ACC), electrodermal activity (EDA), and skin temperature (ST) from 33 people every day for a month as biological data, creates features from the collected biological data, selects a maximum of 10 features by multiple regression analysis to create a stress estimation model, and uses the stress estimation model to calculate a stress evaluation value.

### SUMMARY

Aspects of non-limiting embodiments of the present disclosure relate to outputting stress evaluation values accumulated in an observation period with high accuracy compared to a case of calculating a collective stress evaluation value for a certain relatively long ongoing period.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing device provided with a processor configured to output an evaluation value of stress accumulated in an observation period on a basis of information indicating a first stress feature accumulated up to before the observation period and a second stress feature received during the observation period.

### BRIEF DESCRIPTION OF THE DRAWINGS

Exemplary embodiment of the present disclosure will be described in detail based on the following figures, wherein:

FIG. 1 is a diagram illustrating an exemplary configuration of an information processing system according to an exemplary embodiment of the present disclosure;

FIG. 2 is a block diagram illustrating one example of a control system of an information processing device;

FIG. 3 is a diagram illustrating an example of a user table;

FIG. 4 is a diagram illustrating an example of a biological information table;

FIG. 5 is a diagram for explaining a pulse wave;

FIGS. 6A and 6B illustrate the relationship between stress and a TP value, in which FIG. 6A is a diagram illustrating change in the TP value over a single day in the case of experiencing a relatively high level of stress, and FIG. 6B is a diagram illustrating change in the TP value over a single data in the case of experiencing a relatively low level of stress;

FIGS. 7A and 7B illustrate the relationship between stress and a PP value, in which FIG. 7A is a diagram illustrating change in the PP value over a single day in the case of experiencing a relatively high level of stress, and FIG. 7B is a diagram illustrating change in the PP value over a single data in the case of experiencing a relatively low level of stress;

FIG. 8 is a flowchart illustrating an example of operations by the information processing device when creating a stress estimation model;

FIG. 9 is a flowchart illustrating an example of operations by the information processing device when calculating a stress evaluation value;

FIG. 10 is a table illustrating features used by models and correlation coefficients; and

FIGS. 11A to 11D are diagrams schematically illustrating a way of computing a stress estimation result up to the previous day.

### DETAILED DESCRIPTION

Hereinafter, an exemplary embodiment of the present disclosure will be described with reference to the drawings. Note that in the drawings, structural elements that have substantially the same function are denoted with the same signs, and duplicate description thereof will be reduced or omitted.

### Overview of Exemplary Embodiment

The information processing device according to the exemplary embodiment is provided with a processor that outputs an evaluation value of stress accumulated in an observation period on the basis of information indicating a first stress feature accumulated up to before the observation period and a second stress feature received during the observation period.

“Stress” refers to information indicating an internal state or a psychological state of a person. In the exemplary embodiment, the observation period is set to a single day as an example, and a specific observation period is also referred to as the current day. For example, the single day treated as the observation period may be the day when the stress evaluation value is output or a day farther in the past than the day when the stress evaluation value is output. The stress accumulated on the day of observation includes a first stress that is accumulated over a relatively long period up to the previous day before the day of observation and still remains on the day of observation, and a second stress that is received on the day of observation. The period of accumulating features of stress or data for computing the features up to the previous day before the day of observation may be set appropriately to a relatively long period, such as a week, a month, or six months, that makes it possible to estimate the normal state of the person.

### Exemplary Embodiment

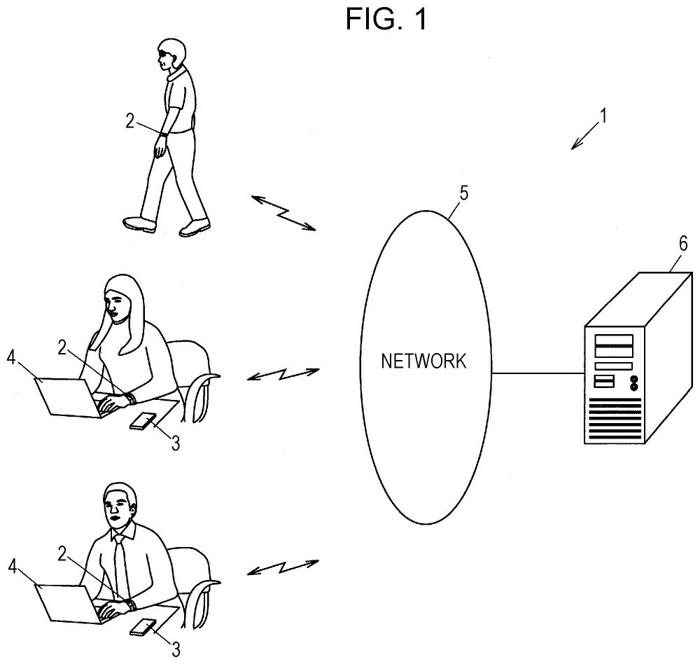

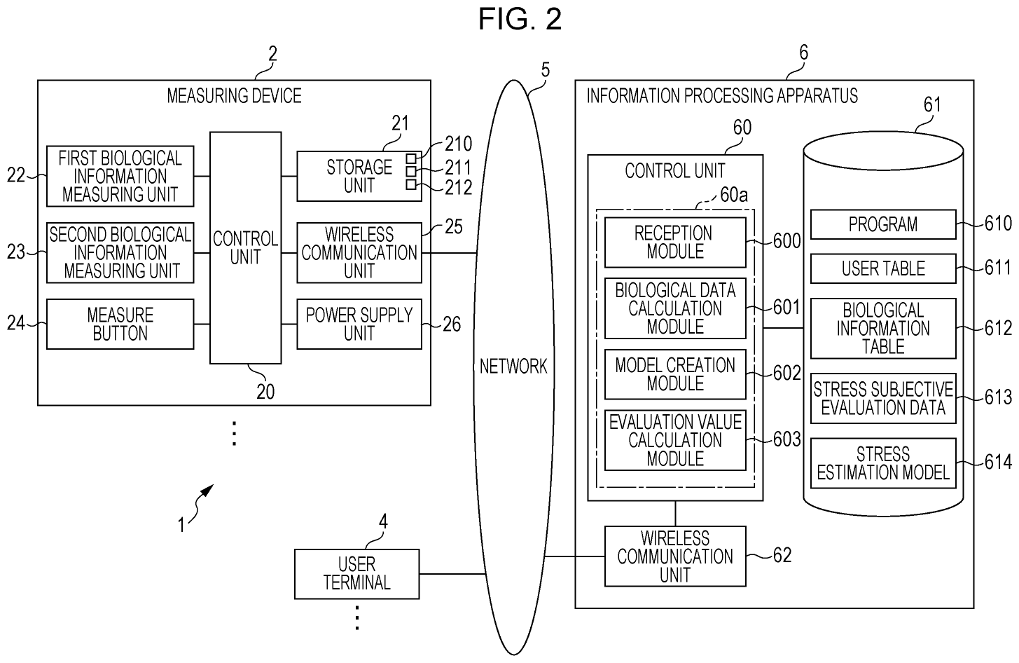

FIG. 1 is a diagram illustrating an exemplary configuration of an information processing system according to an exemplary embodiment of the present disclosure. An information processing system 1 is provided with a measuring device 2 that is worn by a user and measures biological information about the user, a charger 3 for the measuring device 2, a user terminal 4 operated by users including administrators (for example, a person in a position to manage other users), and an information processing device 6 such as a server connected to the measuring device 2 and the user terminal 4 over a network 5. FIG. 1 and FIG. 2 described later illustrate multiple measuring devices 2 and user terminals 4, but it is also possible to have a single measuring device 2 and a single user terminal 4. The user is one example of a measurement subject. The measuring device 2 is one example of a measuring device.

The information processing system 1 is applied to an activity area, which may be a workplace such as an office (including rental offices and shared offices) or a factory, a school, or a place of learning such as a classroom, for example. FIG. 1 illustrates a case of applying the information processing system 1 to an office. The measuring device 2 measures biological information while the user is active in the activity area, for example. Biological information refers to information produced by the body.

When the measuring device 2 is connected to the charger 3, the charger 3 charges a power supply unit 26 described later in the measuring device 2.

For the user terminal 4, a personal computer or a mobile information processing device such as a multifunctional mobile phone (that is, a smartphone) may be used, for example. An IP address is assigned to the user terminal 4.

The network 5 is a communication network such as a wireless local area network (LAN) or the Internet, for example.

FIG. 2 is a block diagram illustrating an example of a control system of the information processing system 1.

(Configuration of Measuring Device)

The measuring device 2 is provided with a control unit 20 that controls each unit of the measuring device 2, a storage unit 21 that stores various information, a first biological information measuring unit 22 that measures first biological information, a second biological information measuring unit 23 that measures second biological information, a measure button 24 that issues an instruction to start and stop measurement, a wireless communication unit 25, and a power supply unit 26 that supplies power to each unit of the measuring device 2.

The control unit 20 includes a processor such as a central processing unit (CPU), an interface, and the like. The functions of the control unit 20 will be described later.

The storage unit 21 includes memory such as read-only memory (ROM) and random access memory (RAM), and stores information such as a program 210 for the processor and user information 211. Also, the storage unit 21 is provided with a biological information storage area 212 where the biological information for a single day is stored. The user information 211 includes information such as a user ID that identifies the user and a measuring device ID that identifies the measuring device 2.

The first biological information measuring unit 22 uses an acceleration sensor, for example. A three-axis acceleration sensor may be used as the acceleration sensor. Hereinafter, time-series data of an acceleration detection signal output by the acceleration sensor is also referred to as acceleration data. The acceleration data is one example of the first biological information.

Note that the first biological information measuring unit 22 may also acquire a movement pattern of a measurement subject on the basis of the detection signal from the acceleration sensor. In this case, a detection signal that acts as a reference for the acceleration sensor is stored in the storage unit 21 for each movement pattern, and the first biological information measuring unit 22 acquires the movement pattern corresponding to the detection signal from the acceleration sensor by referencing the content stored in the storage unit 21. Movement patterns include movements such as sitting, walking, and running, for example.

The second biological information measuring unit 23 uses a pulse wave sensor, for example. An optical pulse wave sensor may be used as the pulse wave sensor. Note that an electrocardio sensor may also be used instead of the pulse wave sensor. Hereinafter, time-series data of a pulse wave signal measured by the pulse wave sensor is also referred to as pulse wave data. The pulse wave data is one example of the second biological information. Information such as the pulse interval and the pulse wave amplitude is acquired from the pulse wave data on the information processing device 6 side, for example. In the case of using an electrocardio sensor, information such as the cardiac interval and the electrocardio amplitude is acquired from the electrocardio data on the information processing device 6 side, for example.

When the measure button 24 is first operated after power-on, the measure button 24 outputs a start signal indicating the start of measurement to the control unit 20, and every time the measure button 24 is operated thereafter, the measure button 24 alternates between outputting a stop signal that indicates the end of measurement and outputting the start signal to the control unit 20.

When the start signal is output from the measure button 24, the control unit 20 controls the first biological information measuring unit 22 and the second biological information measuring unit 23 to start measurement of the first biological information and the second biological information. When the stop signal is output from the measure button 24, the control unit 20 controls the first biological information measuring unit 22 and the second biological information measuring unit 23 to stop measurement of the first biological information and the second biological information.

Also, the control unit 20 stores the first biological information and the second biological information measured between the start signal and the stop signal in the biological information storage area 212 of the storage unit 21. Also, when a predetermined time (for example, 9 PM) is reached, the control unit 20 transmits the first biological information and the second biological information stored in the biological information storage area 212 together with the user information 211 stored in the storage unit 21 to the information processing device 6 over the network 5 using the wireless communication unit 25.

Note that the control unit 20 may also transmit the first biological information and the second biological information measured from a first time (such as a time of arriving at a workplace, a time of taking a seat, a time of starting work duties, or a time when a lecture starts, for example) to a second time (for example, a time of leaving the workplace, a time of leaving the seat, a time of ending work duties, or a time when the lecture ends, for example) in a single workday or day of study to the information processing system 1 at the second time (for example, 6 PM) or at a predetermined time (for example, 9 PM) later than the second time.

The wireless communication unit 25 transmits and receives information with respect to the information processing device 6 over the network 5 using wireless communication such as Bluetooth (registered trademark) or Wi-Fi (registered trademark), for example.

The power supply unit 26 uses a secondary battery such as a lithium-ion secondary battery, for example. Note that a primary battery, a solar cell, or the like may also be used.

(Configuration of Information Processing Device)

The information processing device 6 is provided with a control unit 60 that controls each unit of the information processing device 6, a storage unit 61 that stores various information, and a wireless communication unit 62.

The control unit 60 includes a processor 60a such as a central processing unit (CPU), an interface, and the like. The processor 60a executes a program 610 stored in the storage unit 61 and thereby functions as modules such as a reception module 600, a biological data calculation module 601, a model creation module 602, and an evaluation value calculation module 603. Details about each of the modules 600 to 603 will be described later.

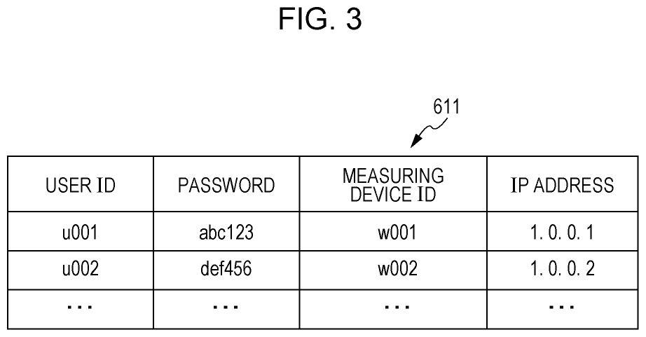

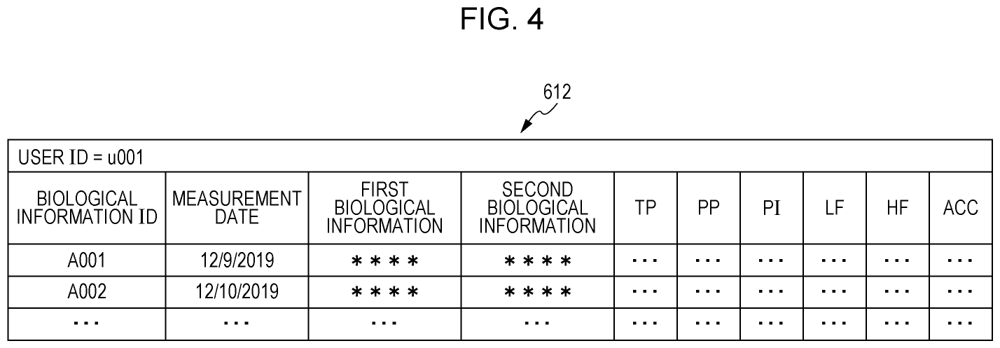

The storage unit 61 includes memory such as read-only memory (ROM), random access memory (RAM), and a hard disk, and stores various information such as the program 610, a user table 611 (see FIG. 3), a biological information table 612 (see FIG. 4), stress subjective evaluation data 613, and a stress estimation model 614. The biological information table 612 is an example of accumulated data. Biological information about multiple users is measured daily and accumulated in the biological information table 612.

The stress subjective evaluation data 613 includes survey results answered by users as a subjective evaluation in response to a stress-related survey (hereinafter also referred to as “a stress subjective evaluation value”), and is stored for each user ID in the storage unit 61. In the stress-related survey, each user answers multiple questions by selecting a degree of stress felt on a five-degree scale.

FIG. 3 is a diagram illustrating an example of the user table 611. The user table 611 includes multiple fields such as a user ID, a password, a measuring device ID, and an IP address. The user ID is an ID that identifies each user. The measuring device ID is an ID that identifies each measuring device 2. The IP address is an IP address assigned to each user terminal 4.

FIG. 4 is a diagram illustrating an example of the biological information table 612. The biological information table 612 is stored for each user ID in the storage unit 61. FIG. 4 illustrates a case where the user ID is “u001”. The biological information table 612 includes multiple fields such as a biological information ID, a measurement date, first biological information, second biological information, TP, PP, PI, LF, HF, and ACC. The user ID is an ID that identifies each user. The biological information ID is an ID that identifies the biological information. The measurement date is the day when the first biological information and the second biological information transmitted from the measuring device 2 is received. In the first biological information field, the acceleration data is recorded. In the second biological information field, the pulse wave data is recorded.

In FIG. 4, TP is an abbreviation of Total Power, and indicates the power of the activity of the autonomic nervous system as a whole as a calculated value obtained by adding together the values for each frequency (hereinafter, the power spectrum) when performing a frequency analysis of the time-series data of the pulse wave interval. PP is an abbreviation of Pulse Pressure, and indicates the pulse wave amplitude that is reflective of the pulse pressure. PI is an abbreviation of Pulse Interval, and indicates the pulse wave interval. LF is an abbreviation of Low Frequency, and indicates the power of the activity of the sympathetic nerves and the parasympathetic nerves as a calculated value obtained by adding together the values of the power spectrum of relatively low frequencies. HF is an abbreviation of High Frequency, and indicates the power of the activity of the parasympathetic nerves as a calculated value obtained by adding together the values of the power spectrum of relatively high frequencies. ACC is an abbreviation of Acceleration, and indicates the acceleration. VLF is an abbreviation of Very Low Frequency, and indicates the overall activity of very slow mechanisms of sympathetic nervous function.

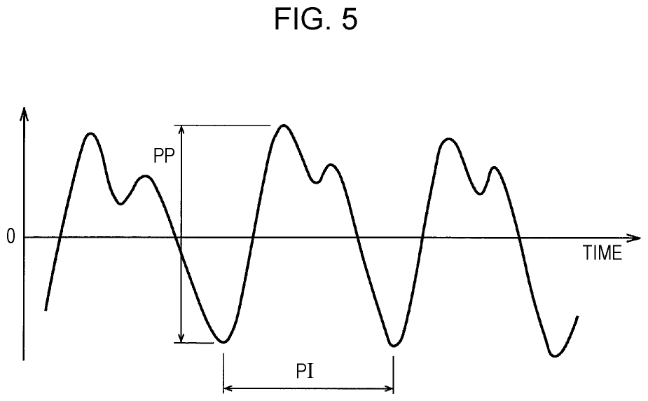

In the TP field, the combined value (hereinafter also referred to as the “TP value”) of the VLF value, the LF value, and the HF value described later is recorded. In the PP field, the pulse wave amplitude (hereinafter also referred to as the “PP value”) (see FIG. 5) is recorded. In the PI field, the pulse wave interval (hereinafter also referred to as the “PI value”) (see FIG. 5) is recorded. In the LF field, the combined value (hereinafter also referred to as the “LF value”) of the power spectrum in the domain of the low-frequency components (hereinafter also referred to as the “LF component domain”) is recorded. In the HF field, the combined value (hereinafter also referred to as the “HF value”) of the power spectrum in the domain of the high-frequency components (hereinafter also referred to as the “HF component domain”) is recorded. In the ACC field, the peak value of the acceleration (hereinafter also referred to as the “ACC value”) is recorded.

The TP value, PP value, PI value, LF value, HF value, and ACC value are calculated by the biological data calculation module 601 on the basis of the acceleration data and the pulse wave data. The TP value, PP value, PI value, LF value, HF value, and ACC value are an example of biological data. Note that the biological data calculation module 601 may also calculate other biological data, such as an LF/HF value.

FIG. 5 is a diagram for explaining a pulse wave. PI indicates the pulse interval, while PP indicates the pulse wave amplitude that is reflective of the pulse pressure. When stress is experienced, PI shortens and PP increases. It is possible to estimate the degree of stress to some extent from information such as PI and PP.

Performing a spectrum analysis of the time-series data of the pulse interval yields the power spectrum. The LF component domain reflects the activity of the sympathetic nerves and the parasympathetic nerves. The HF component domain reflects the activity of the parasympathetic nerves. The LF/HF value indicates the activity of the sympathetic nerves, and serves as an indicator of stress. The sum of the values VLF+LF+HF indicates the total power of the autonomic nervous system as a whole.

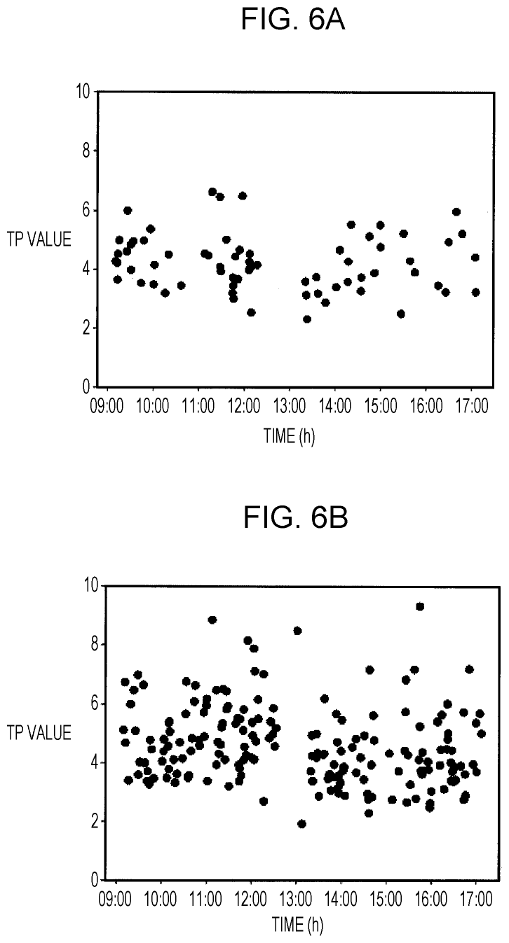

FIGS. 6A and 6B illustrate the relationship between stress and the TP value, in which FIG. 6A is a diagram illustrating change in the TP value over a single day in the case of experiencing a relatively high level of stress, and FIG. 6B is a diagram illustrating change in the TP value over a single data in the case of experiencing a relatively low level of stress. In the case where a relatively high level of stress is experienced, the TP value expressing liveliness is lower, as illustrated in FIG. 6A. In the case where a relatively low level of stress is experienced, the TP value is higher, as illustrated in FIG. 6B.

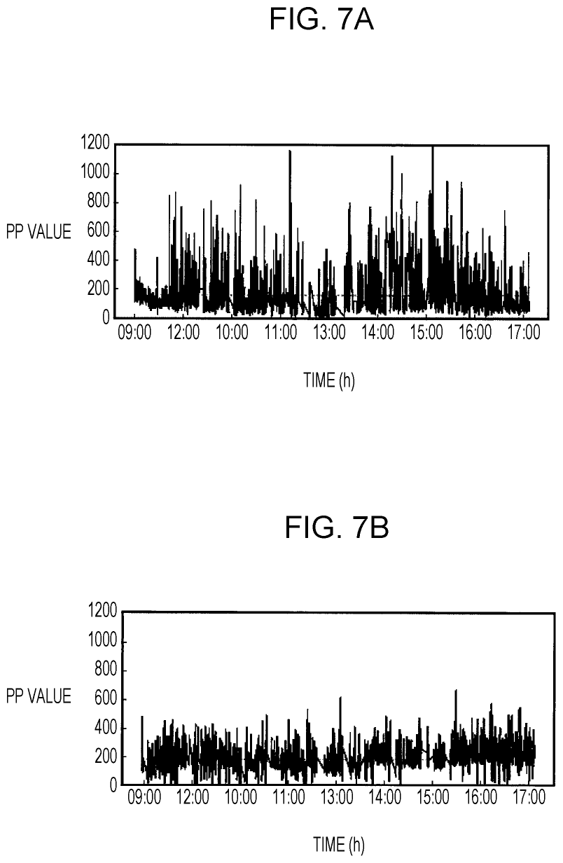

FIGS. 7A and 7B illustrate the relationship between stress and the PP value, in which FIG. 7A is a diagram illustrating change in the PP value over a single day in the case of experiencing a relatively high level of stress, and FIG. 7B is a diagram illustrating change in the PP value over a single data in the case of experiencing a relatively low level of stress. In the case where a relatively high level of stress is experienced, spikes in the PP value occur, as illustrated in FIG. 7A. In the case where a relatively low level of stress is experienced, spikes in the PP value do not occur as much, as illustrated in FIG. 7B.

Next, each of the modules 600 to 603 of the control unit 60 will be described.

When the first biological information (for example, acceleration data), the second biological information (for example, pulse data), and the user information 211 are received from the measuring device 2, the reception module 600 generates a biological information ID, records the biological information ID in the biological information ID field of the biological information table 612 corresponding to the user ID included in the user information 211, records the date when the first biological information and the second biological information are received in the measurement date field, records the acceleration data in the first biological information field, and records the pulse data in the second biological information field.

The biological data calculation module 601 calculates biological data such as the TP value, PP value, PI value, LF value, HF value, and ACC value on the basis of the acceleration data and the pulse wave data recorded in the biological information table 612, and records the calculation results in the corresponding fields of the biological information table 612.

The model creation module 602 transmits the stress-related survey to the user terminals 4 of multiple test subjects over the network 5, receives the survey results responding to the survey transmitted from each user terminal 4, and stores the survey results in the storage unit 61 as the stress subjective evaluation data 613. The model creation module 602 performs multiple regression analysis treating the stress subjective evaluation values included in the stress subjective evaluation data 613 as response variables and features as explanatory variables to create the stress estimation model 614. The model creation module 602 stores the created stress estimation model 614 in the storage unit 61.

When creating the stress estimation model 614, the model creation module 602 specifies the following features, for example. The stress estimation model 614 extracts features correlated with the stress subjective evaluation values from among approximately 400 features, and performs multiple regression analysis to specify nine features effective that are effective for estimating stress. The nine specified features are described next. Note that the features used to estimate stress are not limited to the following nine features.

### (i) Diurnal Difference in Previous Day TP Value

The diurnal difference in the TP value refers to the difference between the maximum value and the minimum value of the TP value in a single day. The TP value expresses the liveliness of the autonomic nervous system, and when a high level of stress is experienced, the autonomic nervous system is exhausted, and the TP value does not take a high value. Exhaustion of the autonomic nervous system on a previous day is not fully recovered, and still remains on the next day. The previous day is one day before the current day. The current day is an example of a day of observation.

### (ii) Ratio of Previous Day PP Value Exceeding Threshold Value

The ratio of the PP value exceeding a threshold value refers to the ratio of the number of times that the PP value exceeds a threshold value in a single day. When stress is experienced, the PP value spikes.

(iii) Diurnal Average Crossing Rate of Previous Day TP Value

The diurnal average crossing rate of the TP value refers to the ratio of the TP value intersecting the average value of the TP value in a single day. When a high level of stress is experienced, this indicator indicates a high value.

### (iv) Diurnal Average Crossing Rate of Current Day TP Value

The diurnal average crossing rate of the TP value has the same meaning as the diurnal average crossing rate of the previous day TP value.

### (v) Diurnal Coefficient of Variation of Current Day TP Value

The diurnal coefficient of variation of the current day TP value is an indicator expressed as the standard deviation divided by the average of the TP value. The diurnal coefficient of variation of the TP value increases with lower levels of stress.

### (vi) Diurnal Different in Current Day TP Value

The diurnal difference in the TP value has the same meaning as the diurnal difference in the previous day TP value.

(vii) Minimum Current Day LF/HF Value

The LF/HF value is the value obtained by dividing the LF value by the HF value. When stress is experienced, the sympathetic nervous system activates, and the LF/HF value rises.

(viii) Diurnal Difference in ACC Value

The diurnal difference in the ACC value refers to the difference between the maximum value and the minimum value of the peaks in acceleration in a single day. When stress is high, sudden movements occur, and the diurnal difference in the ACC value increases.

### (ix) Diurnal Average Crossing Rate of HF Value

The diurnal average crossing rate of the HF value refers to the ratio of the HF value intersecting the average value of the HF value in a single day. The HF value expresses the activity of the parasympathetic nervous system. On a high-stress day, the parasympathetic nervous system is activated and deactivated frequently.

The evaluation value calculation module 603 substitutes the calculated values of the features specified by the model creation module 602 into the stress estimation model 614 created by the model creation module 602, and calculates an evaluation value of the stress of a specific user. In other words, the evaluation value calculation module 603 calculates an evaluation value Eₛₜᵣₑₛₛ of the stress accumulated on the current day by using the stress estimation model 614 expressed in the following Formula (1).

Eₛₜᵣₑₛₛ=(previous day stress features)+(current day stress features)−(current day stress recovery feature)+constant (w₀)

=(w₁x₁+w₂x₂+w₃x₃)+(w₄x₄+w₅x₅+w₆x₆+w₇x₇+w₈x₈)−(w₉x₉)+(w₀) (1)

The previous day stress features are the diurnal difference in the previous day TP value, the diurnal average crossing rate of the previous day TP value, and the ratio of the previous day PP value exceeding the threshold value, for example, and the features are taken to be x₁ to x₃ with coefficients w₁ to w₃, respectively.

The current day stress features are the diurnal average crossing rate of the TP value, the diurnal coefficient of variation of the TP value, the diurnal difference in the TP value, the minimum LF/HF value, and the diurnal difference in the ACC value, for example, and the features are taken to be x₄ to x₈ with coefficients w₄ to w₈, respectively.

The current day stress recovery feature is the diurnal average crossing rate of the HF value, for example, and is taken to be x₉ with a coefficient w₉.

(Operations by Information Processing Device)

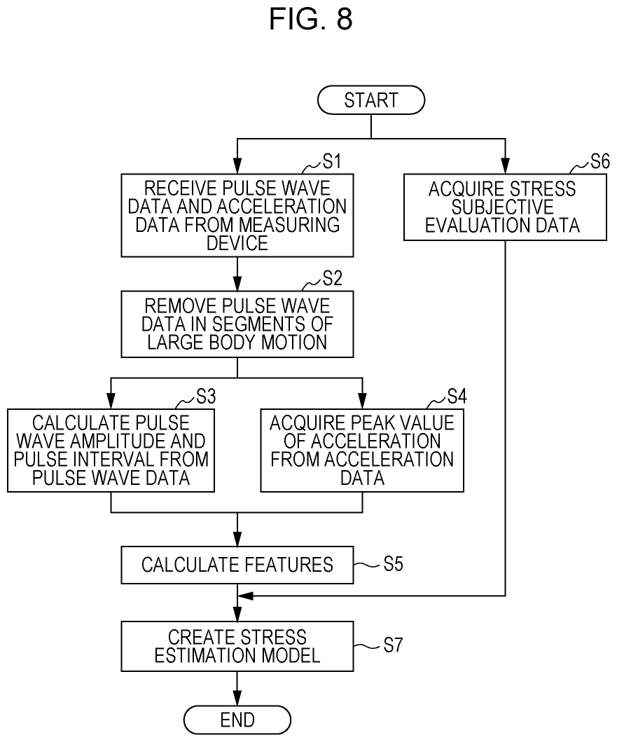

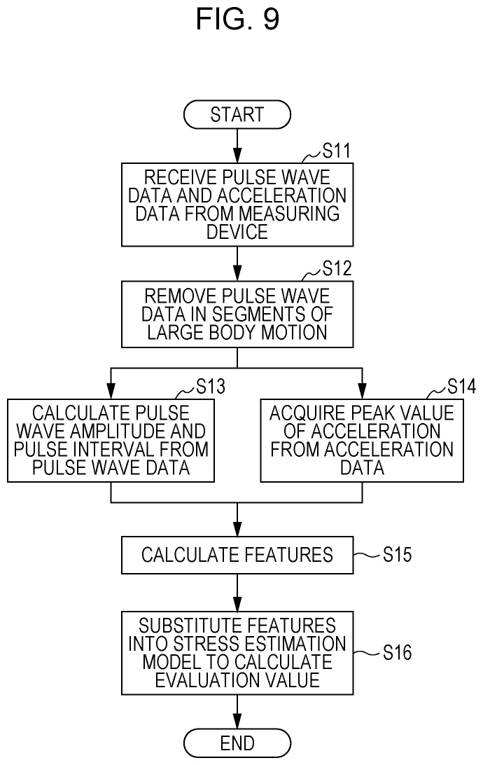

Next, an example of operations by the information processing device 6 will be described with reference to FIGS. 8 and 9. FIG. 8 is a flowchart illustrating an example of operations by the information processing device 6 when creating a stress estimation model. FIG. 9 is a flowchart illustrating an example of operations by the information processing device 6 when outputting a stress evaluation value.

(1) Creation of Stress Estimation Model

The reception module 600 receives the acceleration data, the pulse wave data, and the user information 211 from the measuring device 2 worn by each of multiple users (for example, 18 people) treated as test subjects (S1).

Next, the reception module 600 removes the pulse wave data in segments of large body motion from the received pulse wave data on the basis of the acceleration data (S2). For example, the pulse wave data in segments where the acceleration exceeds a threshold may be removed, or user movement patterns may be estimated on the basis of the acceleration data and the pulse wave data in walking and running segments may be removed.

Next, the reception module 600 generates a biological information ID and records the biological information ID, the measurement date, the acceleration data, and the pulse wave data in the biological information table 612 corresponding to the user ID included in the user information 211. In other words, the biological information ID is recorded in the biological information ID field, the date when the biological information is received is recorded in the measurement date field, the acceleration data is recorded in the first biological information field, and the pulse wave data is recorded in the second biological information field of the biological information table 612.

For example, the biological information and the like of multiple test subjects measured on a workday over a certain period (for example, one week, two weeks, or more) is recorded in the biological information table 612. Note that the biological information may also be measured every day, including days off, over a certain period.

The biological data calculation module 601 calculates the pulse wave amplitude (PP value) and the pulse wave interval (PI value) from the pulse wave data (S3), and acquires the peak value (ACC value) of the acceleration from the acceleration data (S4). Also, the biological data calculation module 601 calculates values such as the TP value, LF value, and HF value in addition to the PP value, PI value, and ACC value. The biological data calculation module 601 records the values such as the TP value, PP value, PI value, LF value, HF value, and ACC value in the corresponding fields of the biological information table 612.

The model creation module 602 references the biological information table 612 and calculates the previous day features x₁ to x₃ and the current day features x₄ to x₉ from the TP value, PP value, PI value, LF value, HF value, and ACC value (S5).

The model creation module 602 acquires the survey results from the test subjects in response to the stress-related survey, and stores the survey results in the storage unit 61 as the stress subjective evaluation data 613 (S6).

The model creation module 602 performs multiple regression analysis treating the stress subjective evaluation values included in the stress subjective evaluation data 613 as response variables and the features x₁ to x₉ as well as the constant w₀ as explanatory variables to create the stress estimation model 614, and stores the created stress estimation model 614 in the storage unit 61 (S7).

(2) Output of Stress Evaluation Value

The reception module 600 receives the acceleration data, the pulse wave data, and the user information 211 from the measuring device 2 worn by a specific user (S11).

Next, as described earlier, the reception module 600 removes the pulse wave data in segments of large body motion from the received pulse wave data on the basis of the acceleration data (S12).

Next, as described earlier, the reception module 600 generates a biological information ID and records the biological information ID, the measurement date, the acceleration data, and the pulse wave data in the biological information table 612 corresponding to the user ID of the specific user included in the user information 211.

The biological information and the like of the specific user treated as the test subject measured on a workday over a certain period (for example, one week, two weeks, or more) is recorded in the biological information table 612 of the specific user. Note that the biological information may also be measured every day, including days off, over a certain period.

The biological data calculation module 601 calculates the pulse wave amplitude (PP value) and the pulse wave interval (PI value) from the pulse wave data (S13), and acquires the peak value of the acceleration from the acceleration data (S14). Also, as described earlier, the biological data calculation module 601 calculates values such as the TP value, LF value, and HF value in addition to the PP value, PI value, and ACC value. The biological data calculation module 601 records the values such as the TP value, PP value, PI value, LF value, HF value, and ACC value in the corresponding fields of the biological information table 612.

The reception module 600 receives a specific day corresponding to the current day from an administrator or the user terminal 4 of the specific user. The evaluation value calculation module 603 references the biological information table 612 corresponding to the user ID of the specific user, calculates the previous day features x₁ to x₃ from the previous day TP value, and calculates the current day features x₄ to x₉ from the current day TP value, LF/HF value, ACC value, and HF value (S15).

The evaluation value calculation module 603 substitutes the features x₁ to x₉ and the constant value w₀ into the stress estimation model 614, and calculates an evaluation value of the stress that the specific user has accumulated on the current day (S16). Note that the stress evaluation value may also be transmitted to an administrator or the user terminal 4 of the specific user.

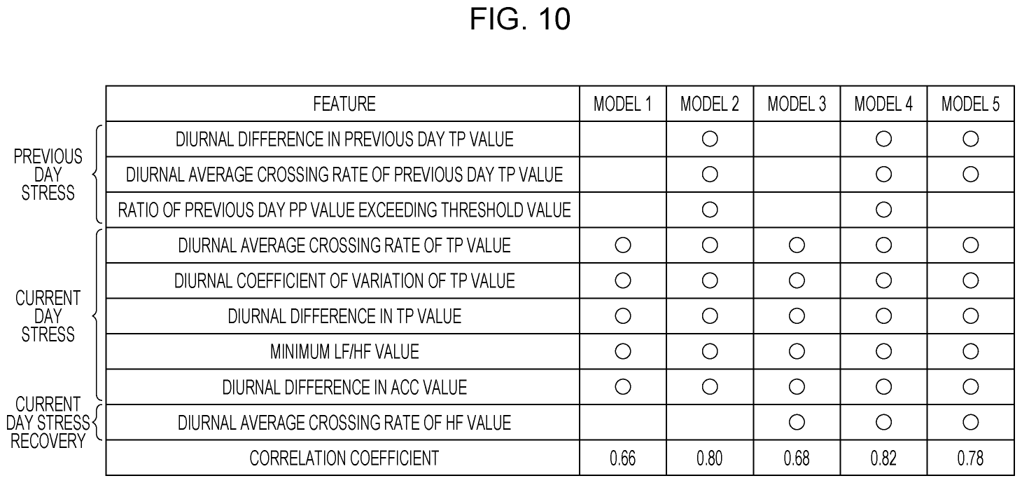

FIG. 10 is a table illustrating features used by models and correlation coefficients. Model 1 is a model that calculates an evaluation value by using only features related to stress on the current day. Model 2 is a model that calculates an evaluation value by using features related to stress on the previous day and the current day. Model 3 is a model that calculates an evaluation value by using features related to stress on the current day and features related to stress recovery on the current day. Model 4 is a model that calculates an evaluation value by using all features, that is, features related to stress on the previous day and the current day and features related to stress recovery on the current day. Model 5 is a model that uses the same features as Model 4 except for the feature of the ratio of the previous day PP exceeding a threshold value.

Model 1 and Model 2 in FIG. 10 demonstrate that by considering the stress on the previous day, the correlation coefficient is improved from 0.66 to 0.80. Similarly, Model 3 and Model 4 demonstrate that by considering the stress on the previous day, the correlation coefficient is improved from 0.68 to 0.82. Also, Model 4 and Model 5 in FIG. 10 demonstrates that by additionally considering the ratio of the previous day PP value exceeding a threshold value from among the stress on the previous day, the correlation coefficient is improved from 0.78 to 0.82. In addition, Model 2 and Model 4 in FIG. 10 demonstrate that by considering stress recovery on the current day, the correlation coefficient is improved from 0.80 to 0.82.

(Exemplary Modification 1)

The exemplary embodiment above calculates the stress evaluation value Eₛₜᵣₑₛₛ by using Formula (1), but the following formula may also be used.

Eₛₜᵣₑₛₛ=(previous day stress estimation result)+(current day stress features)−(current day stress recovery feature)+constant (w₀) (2)

The previous day stress estimation result is obtained by multiplying the previous day stress features (w₁x₁+w₂x₂+w₃x₃) used to calculate the stress evaluation value for the day before the previous day by a coefficient. In the case of using Formula (2), the process of calculating the individual features w₁x₁, w₂x₂, and w₂x₃ may be omitted.

(Exemplary Modification 2)

The exemplary embodiment above calculates the stress evaluation value Eₛₜᵣₑₛₛ by using Formula (1), but the following formula may also be used.

Eₛₜᵣₑₛₛ=(stress features up to current day=features from N days ago+features from (N−1) days ago+ . . . previous day features)+(current day stress features)−(current day stress recovery feature)+constant (w₀) (3)

In the case of using Formula (3), it is possible to compute a more accurate evaluation value compared to the case of using Formula (1).

(Exemplary Modification 3)

The exemplary embodiment above calculates the stress evaluation value Eₛₜᵣₑₛₛ by using Formula (1), but the following formula may also be used.

Eₛₜᵣₑₛₛ=(stress estimation result up to previous day=features from N days ago+features from (N−1) days ago+ . . . previous day features)+(current day stress features)−(current day stress recovery feature)+constant (w₀) . . . (4)

The stress estimation result up to the previous day is obtained by multiplying the features (features from N days ago+features from (N−1) days ago+ . . . previous day features) by corresponding coefficients according to the number of days before the current day. In the case of using Formula (4), the process of calculating the individual features from N days ago, from (N−1) days ago, and so on up to the previous day features may be omitted.

(Exemplary Modification 4)

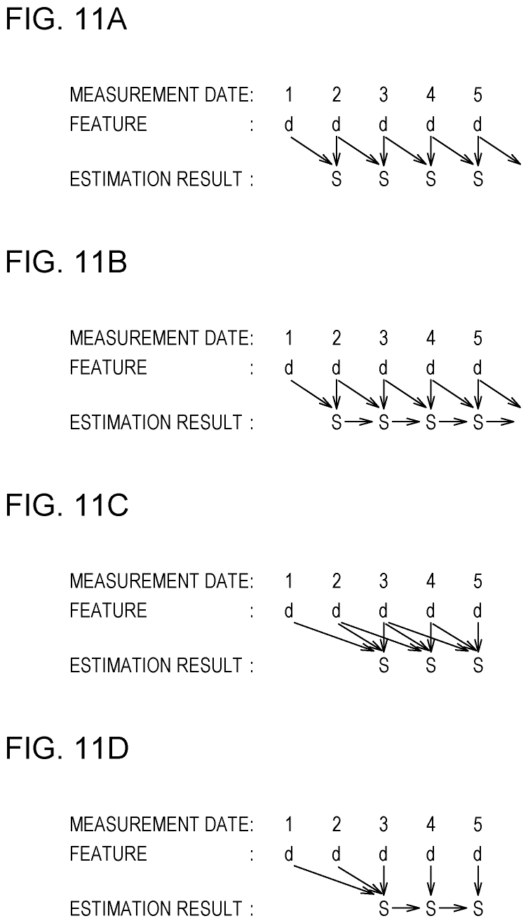

FIGS. 11A to 11D are diagrams schematically illustrating a way of computing a stress estimation result up to the previous day. The stress estimation result up to the previous day may be computed as illustrated in FIGS. 11A to 11D.

For example, as illustrated in FIG. 11A, with respect to the current day (for example, the measurement date is on the 3rd of the month), a stress estimation result S for the previous day may be computed from a feature d from two days ago (for example, the measurement date is on the 1st of the month) and a feature d from the previous day (for example, the measurement date is on the 2nd of the month).

Also, as illustrated in FIG. 11B, for the first stress estimation result for the previous day used in the calculation, the stress estimation result S for the previous day may be computed from the feature d from two days ago (for example, the measurement date is on the 1st of the month) and the feature d from the previous day (for example, the measurement date is on the 2nd of the month), while the stress estimation result for the previous day thereafter may be computed from the stress estimation result S from two days ago (for example, the measurement date is on the 2nd of the month) and the feature d from the previous day (for example, the measurement date is on the 3rd of the month).

Also, as illustrated in FIG. 11C, with respect to the current day (for example, the measurement date is on the 4th of the month), the stress estimation result S for the previous day may be computed from the feature d from three days ago (for example, the measurement date is on the 1st of the month), the feature d from two days ago (for example, the measurement date is on the 2nd of the month), and the feature d from the previous day (for example, the measurement date is on the 3rd of the month).

Also, as illustrated in FIG. 11D, for the first stress estimation result for the previous day used in the calculation, with respect to the current day (for example, the measurement date is on the 4th of the month), the stress estimation result S for the previous day may be computed from the feature d from three days ago (for example, the measurement date is on the 1st of the month), the feature d from two days ago (for example, the measurement date is on the 2nd of the month), and the feature d from the previous day (for example, the measurement date is on the 3rd of the month), while the stress estimation result S for the previous day thereafter may be computed from the stress estimation result from two days ago (for example, the measurement date is on the 3rd of the month) and the feature d from the previous day (for example, the measurement date is on the 4th of the month).

The above describes an exemplary embodiment of the present disclosure, but an exemplary embodiment of the present disclosure is not limited to the foregoing exemplary embodiment, and various modifications are possible within a scope that does not depart from the gist of the present disclosure. For example, to reduce the load on the information processing device 6, all or part of the biological data or the features calculated on the information processing device 6 side may also be calculated on the measuring device 2 side.

Each module of the processor may also be realized by a hardware circuit such as a field-programmable gate array (FPGA) that is partially or fully reconfigurable, or an application-specific integrated circuit (ASIC).

Furthermore, it is also possible to omit or change some of the structural elements of the foregoing exemplary embodiment, within a scope that does not depart from the gist of the present disclosure. In addition, in the flows of the foregoing exemplary embodiment, steps may be added, removed, changed, rearranged, or the like, within a scope that does not depart from the gist of the present disclosure. Also, a program used by the foregoing exemplary embodiment may be provided by being recorded on a computer-readable recording medium such as a CD-ROM, or may be stored on an external server such as a cloud server and used over a network.

In the embodiment above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device).

In the embodiment above, the term “processor” is broad enough to encompass one processor or plural processors in collaboration which are located physically apart from each other but may work cooperatively. The order of operations of the processor is not limited to one described in the embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing device comprising:
a processor configured to
output an evaluation value of stress accumulated in an observation period on a basis of information indicating a first stress feature accumulated up to before the observation period and a second stress feature received during the observation period.

2. The information processing device according to claim 1, wherein
the information indicating the first stress feature is a feature of stress accumulated in a predetermined period before the observation period.

3. The information processing device according to claim 1, wherein
the information indicating the first stress feature is a sum of features of stress respectively accumulated in a plurality of predetermined periods up to before the observation period.

4. The information processing device according to claim 1, wherein
the information indicating the first stress feature is a feature estimated from a feature of stress accumulated in a predetermined period up to before the observation period.

5. The information processing device according to claim 1, wherein
the information indicating the first stress feature includes information related to a total power of autonomic nerves.

6. The information processing apparatus according to claim 5, wherein
the information indicating the first stress feature additionally includes information related to a pulse wave amplitude.

7. The information processing device according to claim 1, wherein
the second stress feature is obtained by subtracting a feature of recovered stress from the stress received in the observation period.

8. The information processing device according to claim 2, wherein
the second stress feature is obtained by subtracting a feature of recovered stress from the stress received in the observation period.

9. The information processing device according to claim 3, wherein
the second stress feature is obtained by subtracting a feature of recovered stress from the stress received in the observation period.

10. The information processing device according to claim 4, wherein
the second stress feature is obtained by subtracting a feature of recovered stress from the stress received in the observation period.

11. The information processing device according to claim 7, wherein
the feature of recovered stress includes information related to a power of parasympathetic nerves.

12. The information processing device according to claim 8, wherein
the feature of recovered stress includes information related to a power of parasympathetic nerves.

13. The information processing device according to claim 9, wherein
the feature of recovered stress includes information related to a power of parasympathetic nerves.

14. The information processing device according to claim 10, wherein
the feature of recovered stress includes information related to a power of parasympathetic nerves.

15. The information processing device according to claim 1, wherein
the processor outputs the evaluation value using a model learned on a basis of accumulated data obtained by measuring and accumulating biological information from a plurality of users.

16. The information processing device according to claim 2, wherein
the processor outputs the evaluation value using a model learned on a basis of accumulated data obtained by measuring and accumulating biological information from a plurality of users.

17. The information processing device according to claim 3, wherein
the processor outputs the evaluation value using a model learned on a basis of accumulated data obtained by measuring and accumulating biological information from a plurality of users.

18. The information processing device according to claim 4, wherein
the processor outputs the evaluation value using a model learned on a basis of accumulated data obtained by measuring and accumulating biological information from a plurality of users.

19. The information processing device according to claim 5, wherein
the processor outputs the evaluation value using a model learned on a basis of accumulated data obtained by measuring and accumulating biological information from a plurality of users.

20. A non-transitory computer readable medium storing a program causing a computer to execute a process for processing information, the process comprising:
outputting an evaluation value of stress accumulated in an observation period on a basis of information indicating a first stress feature accumulated up to before the observation period and a second stress feature received during the observation period.
